(19) United States
(12) Patent Application Publication
(10) Pub. No.: US 2025/0101850 A1
(43) Pub. Date: Mar. 27, 2025

(54) SYSTEM AND METHOD FOR MATCHING BED BOUNDARIES AND DEPTH BETWEEN CORE AND WELL LOGS

(71) Applicant: ARAMCO SERVICES COMPANY, Houston, TX (US)

(72) Inventors: Chicheng Xu, Houston, TX (US); Tao Lin, Katy, TX (US); Zainab Al Ibrahim, Safwa (SA); Yaser Abdullah Alzayer, Dhahran (SA); Weichang Li, Katy, TX (US); Lei Fu, Houston, TX (US)

(73) Assignee: ARAMCO SERVICES COMPANY, Houston, TX (US)

(21) Appl. No.: 18/474,986

(22) Filed: Sep. 26, 2023

(51) Int. Cl. E21B 44/00; G01N 33/24; G01V 5/06; G06T 7/32

(52) U.S. Cl. CPC E21B 44/00; G01N 33/24; G01V 5/06; G06T 7/32; E21B 2200/20; G06T 2207/10024; G06T 2207/20072; G06T 2207/30181

(57) ABSTRACT

A method may use a core sampling system for collecting a core sample with a reference log of a first property. The method may use a wellbore logging system for recording uncalibrated well logs with a target log of the first property. The method may use a computer processor for obtaining an uncalibrated geological model, determining a bulk-shift depth correction based on a first cost function, forming a bulk-shifted log by applying the bulk-shift depth correction to the target log, identifying a plurality of log event pairs, determining, for each of the log event pairs, a local-shift depth correction based on a second cost function, forming a local-shift depth correction table from the local-shift depth correction for the log event pairs, and forming a calibrated geological model based, at least in part, on the uncalibrated geological model and the local-shift depth correction table.

### BACKGROUND

In the oil and gas industry wells, hydrocarbons are located in reservoirs far beneath the surface of the Earth. Wells are drilled into subsurface reservoirs to access and produce the hydrocarbons. As a wellbore is created beneath the surface of the Earth, rock core samples are often extracted and brought to the surface for examination and analysis. In conventional coring, a cylindrical section of rock is cut and removed from the path of the wellbore by a coring bit. Once extracted, core samples are often examined to determine reservoir characteristics. A reservoir characteristic may incorporate any of the characteristics pertinent to the reservoir's ability to store and produce hydrocarbons.

Information about the subsurface, often represented in the form of digital models, is used to evaluate the reservoir. Information is combined from many sources: remote sensing e.g., seismic, gravity, direct sampling, e.g., core samples from current and previous wells, and proximate measurements, e.g., well logs. A recurrent problem is ensuring that each of these disparate datasets are depth calibrated or matched.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

It is intended that the subject matter of any of the embodiments described herein may be combined with other embodiments described separately, except where otherwise contradictory.

This disclosure presents, in accordance with one or more embodiments, a method that includes collecting a core sample using a core sampling system. The core sample includes a reference log of a first property. The method includes recording a plurality of uncalibrated well logs using a wellbore logging system. The plurality of uncalibrated well logs includes a target log of the first property. The method includes using a computer processor for obtaining an uncalibrated geological model, then determining a bulk-shift depth correction based on a first cost function formed from the reference log and the target log. The method includes using the computer processor for forming a bulk-shifted log by applying the bulk-shift depth correction to the target log, then identifying a plurality of log event pairs. Each of the log event pairs includes an event on the bulk-shifted log and a corresponding event on the reference log. The method includes using the computer processor for determining, for each of the log event pairs, a local-shift depth correction based on a second cost function formed from the reference log and the bulk-shifted log over a depth-window surrounding the log event pair. The method includes using the computer processor for forming a local-shift depth correction table from the local-shift depth correction for the log event pairs, then forming a calibrated geological model based, at least in part, on the uncalibrated geological model and the local-shift depth correction table.

This disclosure presents, in accordance with one or more embodiments, a system that includes a core sampling system configured to collect a core sample. The core sample includes a reference log of a first property. The system includes a wellbore logging system configured to record a plurality of uncalibrated well logs. The plurality of uncalibrated well logs includes a target log of the first property. The system includes a computer processor configured to obtain an uncalibrated geological model, then determine a bulk-shift depth correction based on a first cost function formed from the reference log and the target log. The system includes a computer processor configured to form a bulk-shifted log by applying the bulk-shift depth correction to the target log. The system includes a computer processor configured to identify a plurality of log event pairs. Each of the log event pairs includes an event on the bulk-shifted log and a corresponding event on the reference log. The system includes a computer processor configured to determine, for each of the log event pairs, a local-shift depth correction based on a second cost function formed from the reference log and the bulk-shifted log over a depth-window surrounding the log event pair. The system includes a computer processor configured to form a local-shift depth correction table from the local-shift depth correction for the log event pairs, then form a calibrated geological model based, at least in part, on the uncalibrated geological model and the local-shift depth correction table.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIG. 1 depicts a petroleum system in accordance with one or more embodiments.

FIG. 2 depicts a core sampling system and a wireline logging system in accordance with one or more embodiments.

FIG. 3 depicts a system block diagram in accordance with one or more embodiments.

FIG. 4A and FIG. 4B depict a log comparison in accordance with one or more embodiments.

FIG. 5 depicts a flowchart in accordance with one or more embodiments.

FIG. 6 depicts an example gamma-ray log in accordance with one or more embodiments.

FIG. 7 depicts an example bed boundary match in accordance with one or more embodiments.

FIG. 8 depicts an example color shift in accordance with one or more embodiments.

FIG. 9 depicts an example well log track in accordance with one or more embodiments.

FIG. 10 depicts an example core photo in accordance with one or more embodiments.

FIG. 11 depicts an example pixel array in accordance with one or more embodiments.

FIG. 12 depicts an example event match in accordance with one or more embodiments.

FIG. 13 depicts an example depth shift table in accordance with one or more embodiments.

FIG. 14 depicts a pre-shift correlation in accordance with one or more embodiments.

FIG. 15 depicts a post-shift correlation in accordance with one or more embodiments.

FIG. 16 depicts a drilling system in accordance with one or more embodiments.

FIG. 17 depicts a computer system in accordance with one or more embodiments.

FIG. 18 depicts a flowchart in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

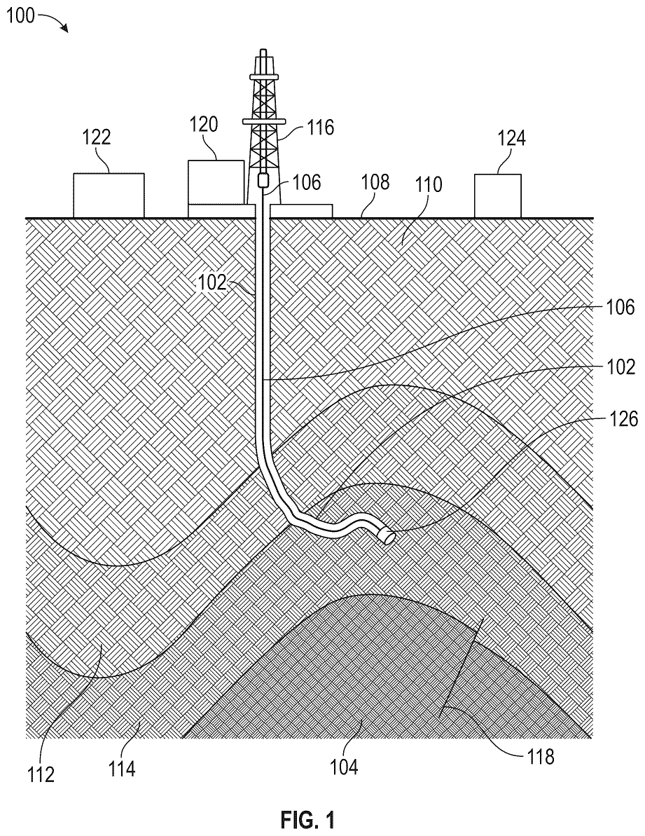

In the following description of FIGS. 1-18, any component described with regard to a figure, in various embodiments disclosed herein, may be equivalent to one or more like-named components described with regard to any other figure. For brevity, descriptions of these components will not be repeated with regard to each figure. Thus, each and every embodiment of the components of each figure is incorporated by reference and assumed to be optionally present within every other figure having one or more like-named components. Additionally, in accordance with various embodiments disclosed herein, any description of the components of a figure is to be interpreted as an optional embodiment which may be implemented in addition to, in conjunction with, or in place of the embodiments described with regard to a corresponding like-named component in any other figure.

It is to be understood that the singular forms “a,” “an,” and “the” include plural referents unless the context clearly dictates otherwise. Thus, for example, reference to “a core sample” includes reference to one or more of such core sample.

Terms such as “approximately,” “substantially,” etc., mean that the recited characteristic, parameter, or value need not be achieved exactly, but that deviations or variations, including for example, tolerances, measurement error, measurement accuracy limitations and other factors known to those of skill in the art, may occur in amounts that do not preclude the effect the characteristic was intended to provide.

It is to be understood that one or more of the steps shown in the flowcharts may be omitted, repeated, and/or performed in a different order than the order shown. Accordingly, the scope disclosed herein should not be considered limited to the specific arrangement of steps shown in the flowcharts.

Although multiple dependent claims are not introduced, it would be apparent to one of ordinary skill that the subject matter of the dependent claims of one or more embodiments may be combined with other dependent claims.

Wells drilled for hydrocarbon exploration or production purposes are commonly logged by lowering a combination of physical sensors downhole by wireline or logging while drilling (LWD) technology to acquire data that measures various rock and fluid properties. Well log data is commonly used to estimate reservoir properties including porosity, fluid saturation, and permeability, which are required for reservoir modeling, reserves estimation, and production forecasting. In some key wells, rock samples or core samples are also taken from the borehole through coring while drilling operations.

For reservoir description, core data is often used to calibrate and verify well-log interpretation based petrophysical models. However, core depth and well log depth are easily mismatched due to their different operational conditions. Core samples are typically indexed by a depth determined by a driller, i.e., a “driller's depth.” Depth is determined differently for cores and wireline logs which may lead to difficulty when they are combined to form petrophysical models. Typically, driller's depth are determined by summing the lengths of segments (or “joints”) of drill pipe suspended from the drilling rig top-drive corrected by the height of the top-drive above a reference level such as the drilling rig floor. In contrast, wireline logs are indexed by a depth determined by a wireline logger, i.e., a logger's depth, and are based upon the length of wireline unspooled into the well during well logging operations.

The differences between logger's depth and driller's depth may be mismatched due to errors in both measurements. This depth mismatch is sometimes referred to as a core shift. In order to enable accurate core measurement calibration for a log-based model, the depth indices for the core and log must be aligned, which can be a tedious, labor-intensive, error-prone process. Accordingly, there exists a need for an intelligent and automatic workflow to improve the efficiency of matching core depth with log depth.

Embodiments disclosed herein relate to a workflow using intelligent algorithms that automatically match core depth with log depth by identifying a series of events on a log curve and matching the events using a local maximum correlation standard, thereby replacing work performed manually. Replacing the manual work improves efficiency of the matching of core depth to log depth. An example of a series of events may include events comprising an extremum of a depth derivative of a first property. The extremum of the depth derivative may be determined, for example, using a maximum variance method. The maximum variance method calculates the variance of a log curve in a sliding window and the variance peaks indicate the boundaries between different rock layers or beds. Consequently, the workflow disclosed herein forms an improvement over the conventional method.

The rock samples or core samples taken from the borehole may have investigations performed on them. Investigations such as photography and physical measurements may be performed on the core samples, including:
Core photo (white light and ultraviolet);
Core plug measurements: routine and special core analysis; and
Core scans: gamma ray (GR), CT, geomechanical scratching test.

Indexing the core samples by a drilling depth may be calculated from the number and length of drill pipes used measured from the surface. Indexing the wireline logs by a logger's depth may be calculated by the depth at the surface under the consideration of the cable configuration.

The core shift, i.e., the differences between logger's and driller's depths are due to the different stretch in the drill string when drilling and the wireline entered into the bore hole during wireline logging operations. In order to enable accurate core measurement calibration for a log-based model, the depth indices for the core and log must be aligned.

Depth matches between core and well logs are typically performed by correlating core GR scans and GR well log measurements. By comparing core GR and log GR, bulk shift can be easily identified and calculated. However, a bulk shift is not always sufficient to achieve the best match. There are often depth mismatches between rock layers on core samples and well logs due to several operational factors, such as:
Yo-Yo™ effect on wireline log
vibration on LWD
core stretching due to stress conditions (unloading)
core damage during transportation or handling

Typical detailed depth matching is done by manually picking a series of peaks or troughs on two different logs and then matching peak to peak or trough to trough. Once a set of depths on both logs are matched, a depth shift table will be created to convert the depth on target log to the depth of the reference log.

The conventional depth matching procedure has several challenges. First, thick and homogeneous or extremely heterogeneous formations do not show distinct peaks or troughs on logs making the manually picking method difficult. Second, it is difficult to apply the depth shift table on core photos. Third, the whole procedure is manually done and labor-intensive.

FIG. 1 depicts a petroleum system 100 in accordance with one or more embodiments. The petroleum system 100 includes a source rock formation 104, or rocks in which hydrocarbons have been generated or are capable of hydrocarbon generation. A source rock formation 104 is one of the necessary elements of the petroleum system 100 and includes organic-rich sediments that may be deposited in a variety of environments. When these organic-rich sediments are heated sufficiently, hydrocarbons may be created. With hydrocarbons present in a source rock formation 104 the petroleum system 100 requires a migration of these hydrocarbons into a hydrocarbon reservoir formation (e.g., reservoir 114). The hydrocarbons may migrate vertically through faults (e.g., fault 118) or fractures upwards into the reservoir 114. The fault 118 depicted in FIG. 1 fractures and displaces the source rock formation 104, allowing for the escape of the hydrocarbons in these cases. Hydrocarbon migration may also occur as a near-vertical migration from the reservoir 114 due to a buoyancy-driven flow of hydrocarbons. A buoyancy-driven flow of hydrocarbons occurs when the upward buoyancy force of the hydrocarbons is greater than the downward force of gravity, causing an upward migration. Migration may be local, as shown in the petroleum system 100 of FIG. 1 or may occur over distances of hundreds of kilometers in larger sedimentary basins and is a crucial mechanism to the formation of a viable petroleum system.

The reservoir 114 contains an accumulation of hydrocarbons including oil and/or natural gas. The reservoir 114 is usually a permeable and porous rock layer capable of storing and transmitting hydrocarbon fluids. Reservoirs (such as reservoir 114) are formed under temperature conditions that may preserve the hydrocarbons and are overlain by an impermeable layer or layers of rock, known as a hydrocarbon seal (e.g., seal 112). The seal 112 acts as a barrier to stop the further migration of hydrocarbons and may be accompanied with an appropriate topographic structure, such as an anticline which helps to further trap the accumulation of hydrocarbons within the reservoir 114. By understanding the regional relationships between the play risk elements of the petroleum system 100, which include information regarding the source rock formation 104, the reservoir 114, the seal 112 and the hydrocarbon charge, the veracity of PBE may be improved. The hydrocarbon charge describes a likelihood of forming hydrocarbons of a petroleum system 100, migrating, and being trapped in the reservoir 114 for economic extraction.

A hydrocarbon exploration may be conducted on a prospective petroleum system by drilling into the suspected reservoir (e.g., reservoir 114) in order to detect, quantify, or extract the hydrocarbons. A wellbore 102 may be drilled by a drill bit 126 attached by a drill pipe 106 to a drill rig 116 located on the surface 108 of the Earth. The wellbore 102 may traverse a plurality of overburden layers 110 and one or more seal formations (e.g., seal 112) to a prospective hydrocarbon reservoir (e.g., reservoir 114). The wellbore 102 may be drilled to perform any number of tests to analyze and broaden the knowledge and understanding of the hydrocarbon characteristics of the reservoir 114, including determining well logs from a logging system 120.

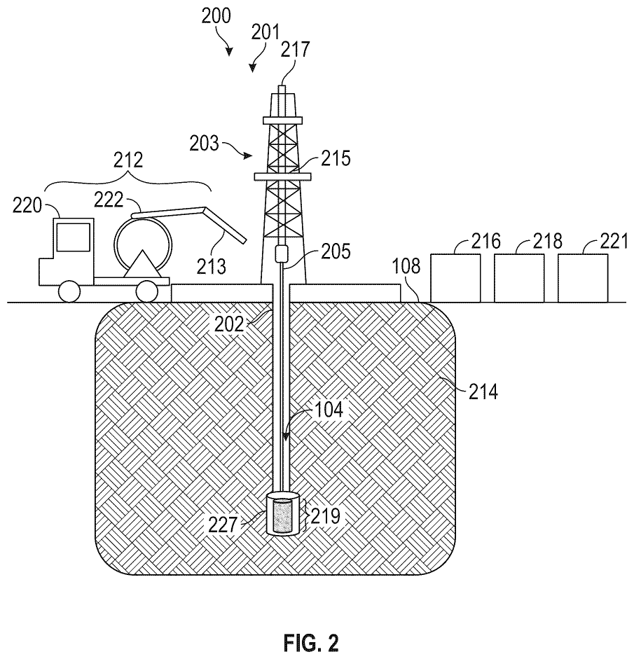

The logging system 120 may include the tools to determine a well log, including tools from a wireline logging operation or a logging while drilling (LWD) operation. A logging tool may be lowered into the wellbore 102 to acquire measurements as the tool traverses a depth interval. The plot of the logging measurements versus depth may be referred to as a log or well log. For example, a logging tool may generate the energy (acoustic, nuclear, magnetic, electrical) that is transmitted into the formation. The logging tool may record the results of the log. The logging tool may store the results of the log and may transmit the results back to the surface. A well log from a logging tool may be termed a logging tool log. Well logs may provide depth measurements of the wellbore 102 that describe such reservoir characteristics as formation porosity, formation permeability, resistivity, water saturation, and the like. The resulting logging measurements may be stored or processed or both, for example, by a control system 122, to generate corresponding well logs for the wellbore 102. The logging tool may have a communication interface for receiving a command signal to begin recording the log, for example a command signal sent from the control system. The logging tool communication interface may be used for transmitting the logging data to the control system 122. The logging tool log may be acquired by, for example, a gamma ray logging tool. The gamma ray logging tool may be configured to acquire the gamma ray log upon receipt of a signal, such as a command signal, to begin logging. Furthermore, acquisition of the gamma ray log may commence after receipt of the signal to begin logging. A control system such as the control system 122 may send the command to begin logging. A well log and how it may be used to determine a depth interval containing a petroleum system element is explained in greater detail in FIG. 2.

Well test results 124, shown as a box in FIG. 1, may be taken from the wellbore 102 during the drilling or completion of the wellbore 102 and may include analyzing any available hydrocarbon show data. Hydrocarbon show data is any indication of oil and gas witnessed during well operations, including any hydrocarbons directly observed in drilling fluids, core samples, and cutting samples. Hydrocarbon show data may also include direct formation fluid samples taken at multiple intervals of the wellbore 102. Formation fluid samples or reservoir fluid samples may be retrieved and analyzed at the surface 108 during a well's production, or fluid recovery devices may be used during the drilling of the well to collect these fluid samples. These fluid samples may be analyzed, usually at a separate location such as a laboratory, to characterize the fluid composition to determine the type of fluid present. These fluid samples are analyzed to identify the presence of oil, gas, water, and combinations of multiple fluid types.

Well test results 124 may also include formation flow tests, which include taking several measurements while the formation fluids travel up the wellbore 102 to be collected. During the formation flow tests, the reservoir fluids are often channeled through a flowmeter (not shown) to determine a formation pressure, a hydrocarbon flow rate, and a fluid characterization. A hydrocarbon flow rate may be characterized as the volume of fluid that moves through a given cross-sectional area per unit time and is usually measured by a flowmeter. Determining a hydrocarbon flow rate may aid in determining certain reservoir formation characteristics such as the volume of hydrocarbons present in the reservoir 114 and the permeability of the formation. Permeability describes the ability for fluids to pass through the formation and is crucial in determining the quality of the reservoir 114 present. These well test results 124, which may include any sampled fluids or a hydrocarbon flow rate, may be used in combination with well logs to determine a petroleum system description for a particular reservoir depth interval. The above petroleum system descriptions may describe hydrocarbon test and show data in addition to seal and source rock quality determined based, at least in part, on the well logs and the well test results 124. Further, the petroleum system descriptions are shown on a visual representation in FIG. 1, which helps to understand a regional petroleum system (e.g., petroleum system 100).

While a single wellbore (e.g., wellbore 102) is illustrated in the petroleum system 100, hydrocarbon exploration and/or production typically involves a plurality of wellbores positioned at various locations of the reservoir 114. By collecting all the available data from the plurality of wellbores, including any well logs and the well test results 124, a greater understanding of the petroleum system 100 may be developed. Decisions, including determining a new drilling target location, may be made based, at least in part, on this increased understanding of the petroleum system 100. For example, well log data are commonly used to estimate reservoir properties including porosity, fluid saturation, and permeability, which are required for reservoir modeling, reserves estimation, and production forecasting. In some key wells, rock samples (e.g., the core 219) are also taken out of the borehole through a coring while drilling operation.

FIG. 2 shows a core sampling system and a logging system in accordance with one or more embodiments. A geological model is used to represent the earth. The geological model may be obtained using seismic data. Seismic data may be subject to depth errors such as the core shift. A core sampling system 201, in accordance with one or more embodiments, may include, for example, a coring rig, a conveyance such as wireline or drill pipe, a core bit, an inner barrel, and an outer barrel. After a well 202 is drilled, core samples may be taken using the core sampling system 201 and wireline logs may be run using a wellbore logging system. The wellbore logging system may produce a wireline well log that may be uncalibrated, e.g., an uncalibrated well log or uncalibrated well logs. Uncalibrated well logs without core samples may be used to form an uncalibrated geophysical model. The core samples and the uncalibrated well logs may be integrated to produce a calibrated geophysical model. The calibrated geophysical model may then be used to define drilling targets.

A method for taking a core sample includes use of a drilling system 203. The drilling system may include the well 202 and a coring bit 227 attached by a drillstring 205 to a coring rig 217. The formation (e.g., reservoir 214) may be cored to produce rock core samples (e.g., core 219) for analysis. Coring operations may include physically extracting a core sample from a region of interest within a wellbore 204 by the coring bit and bringing it to the surface 108 for examination. A second coring technique, termed sidewall coring, may also be used to extract a core sample. Two techniques are used for obtaining sidewall cores. In rotary sidewall coring, mechanical tools may use hollow rotary drills to cut through the sidewall rock formation producing rotary sidewall cores. In percussion sidewall coring a tool uses a propellant to shoot a hollow, retrievable, cylindrical bullet into the wellbore wall. For example, the percussion sidewall coring and rotary side wall coring may be discrete depth indexed in separate runs. To align their depth to the reference log in a particular run may include aligning the two Gamma ray logs acquired in the two runs. Following development of a depth shift table, the depth shift values may be applied to the depths of sidewall coring points.

The core samples (e.g., core 219), usually cylindrical, may be analyzed in a laboratory to determine various reservoir characteristics from the location from which the sample was obtained. These reservoir characteristics may include porosity, pore size distribution, permeability, or the presence of hydrocarbons. Porosity may indicate how much void space or pore space exists in a particular rock within the formation (e.g., reservoir 214), where oil, gas or water may be trapped. Pore size distribution describes the relative abundance of each pore size in a particular rock and permeability may indicate the ability of liquids and gases to flow through the rock within the area of interest.

Core samples are obtained from rock that is under compressive stress. Upon removal of a core from its source rock, the core is relieved of the compressive stress and therefore the core may expand. This relief of compressive stress is termed unloading. The core sample unloading is one of the operational factors that leads to the depth mismatch.

The wellsite 200 may include a control system 216, having hardware and/or software for managing drilling operations, logging operations and/or maintenance operations. For example, the control system may include one or more programmable logic controllers (PLCs) that include hardware and/or software with functionality to control one or more processes performed by the drilling system 203. The wellsite may also include a logging system 212 with one or more logging tools (e.g., a logging tool 213), such as a gamma ray logging tool and a nuclear magnetic resonance (NMR) logging tool for use in generating well logs of the formation. Following the removal of the drilling system, the logging system may be lowered into the wellbore to acquire measurements as the tool traverses a depth interval. The plot of the logging measurements versus depth may be referred to as a log or well log. Well logs may provide depth measurements of the well that describe reservoir characteristics including formation porosity, formation permeability, resistivity, water saturation, and the like. The resulting logging measurements may be stored or processed or both, for example, by the control system 216, to generate corresponding well logs for the well. The control system 122 or the control system 216 may send the command to the logging tool to begin logging. The logging system may be supported by a truck 220 and derrick 215 above ground. For example, the truck may carry a conveyance mechanism 222 used to lower the logging tools into the wellbore. The conveyance mechanism may be a wireline, coiled tubing, or drill pipe that may include means to provide power to the well logging system and a telemetry channel from the well logging system to the surface. In some embodiments, the well logging system may be translated along the depth of the wellbore to acquire a well log over multiple depth intervals.

The logging tools may include, but are not limited to caliper, electrical resistivity, resistivity image, electromagnetic, acoustic logging tools, acoustic image logging tools, neutron, and gamma ray logging tools. Thus, the well log acquired from the well logging system may be an acoustic log, acoustic image log, electrical resistivity, or resistivity image log. For example, a gamma ray log measures radioactivity found naturally in the formation. In the case of a gamma ray log, the log measures a gamma-ray emission property of the logged area such as rock, core, wellbore, borehole, or formation. A term of art is eLog which refers to electrical wireline log. Another example is an NMR logging tool which measures the induced magnetic moment of hydrogen nuclei (specifically, protons) contained within the fluid-filled pore space of porous media (for example, reservoir rocks). Various logging tools may measure porosity, permeability, pore size, pore-size distribution and the types of fluids present in the pore spaces. These reservoir characteristics are pertinent for reservoir characterization.

Logs obtained by wireline may contribute to the depth mismatch due to the Yo-Yo effect. The wireline has an inherent stretch property. If or when the logging tool sticks or drags in the bore and then frees itself, the tool may travel up and down the wellbore, oscillating within a certain distance range. The oscillation range narrows as the Yo-Yo effect diminishes following a roughly second-order decay. At a point in time the logging tool stabilizes. Prior to the stabilization, depth records of the wireline tool may have a depth mismatch with respect the retrieved core.

Logs obtained by LWD may contribute to the depth mismatch due to vibration of the drilling operation. As the drill bit drills into the rock, the drill bit may vibrate in several axes. A logging tool coupled to the drill bit may record depth records that have a depth mismatch with respect to the retrieved core.

Core damage may contribute to the depth mismatch. Upon retrieval, the core integrity may be disturbed due to handling, packaging, and transportation. Disturbing the core integrity may result in core damage. For example, core damage may be generalized as improper stabilization of unconsolidated samples prior to shipping; time lag may exceed a time envelope for determining saturations; and lack of comprehensive information on the sample container or the accompanying data sheet may lead to improper handling and subsequent core damage. A damaged core may result in a depth mismatch with respect to the logged depth data. The core sample may be evaluated using gamma-ray logging, such as detecting a gamma-ray emission. The core sample may be evaluated by imaging the core sample such as by taking a photographic image of each core sample. The photographs may have pixels in colors of, for example, red, green, and blue. The photographs may produce images (e.g., a core image) that show various features such as peaks, troughs, and bed boundaries. A bed is a layer of sediment or sedimentary rock, i.e., a stratum. Bed boundaries are the boundaries between two different rock types. To be labeled a bed, the stratum must be distinguishable from adjacent beds. A bed boundary is the interface between adjacent beds of sedimentary rock layer or stratum. The boundary separates each stratum, where a bed is the smallest stratigraphic unit, in a succession of strata bounded by unconformities or their correlative conformities. A key property of each stratum is that they are chronostratigraphically significant surfaces. Thus, each surface is a physical boundary that separates all the rocks above from those below. The features visible in the images may correspond with log events such as gamma-ray emissions that may be paired with log events obtained using a core sample log or a wireline log to form log event pairs. The depths of the core sample log events may not match the depths of the wireline log (or tool log) depths. This mismatch may be compensated for by shifting the core image to more closely match the depth of the logged depth. This core shift, when applied to the core image, may be referred to as a depth-shifted core image. All logs can be used to identify bed boundaries, but gamma ray is the most commonly available log between core and log or between wells.

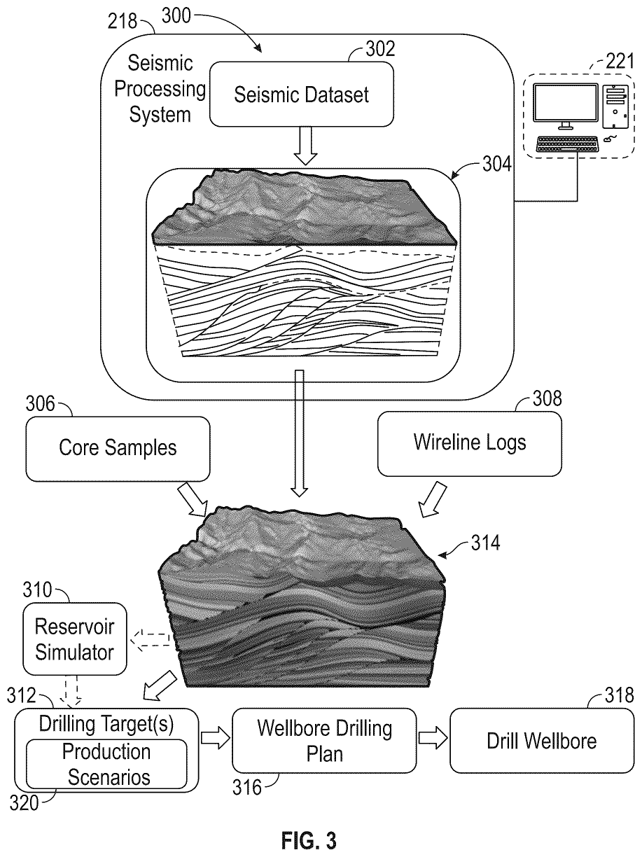

FIG. 3 depicts a system block diagram in accordance with one or more embodiments. In some embodiments, a well planning system 300 uses a seismic dataset 302 as an input to such as an uncalibrated geological model 304. Using core samples (e.g., a core sample 306) and wireline logs 308 the uncalibrated geological model 304 may be calibrated to form a calibrated geological model 314. Using the well planning system, the calibrated geological model 314 may be used in the reservoir simulator 310 and/or it may be used directly to set drilling targets (e.g., drilling target 312) as a component of a wellbore drilling plan (e.g., wellbore plan 316). Such a wellbore plan may contain the drilling targets (e.g., a drilling target 312): geological regions expected to contain hydrocarbons. The well planning system 300 may plan wellbore trajectories to reach the drilling targets while simultaneously avoiding drilling hazards, such as preexisting wellbores, shallow gas pockets, and fault zones, and not exceeding the constraints, such as torque, drag, and wellbore curvature, of the drilling system. Similarly, the wellbore plan may include a determination of wellbore caliper and casing points. In this manner the drilling system may drill the wellbore guided by the planned wellbore trajectory.

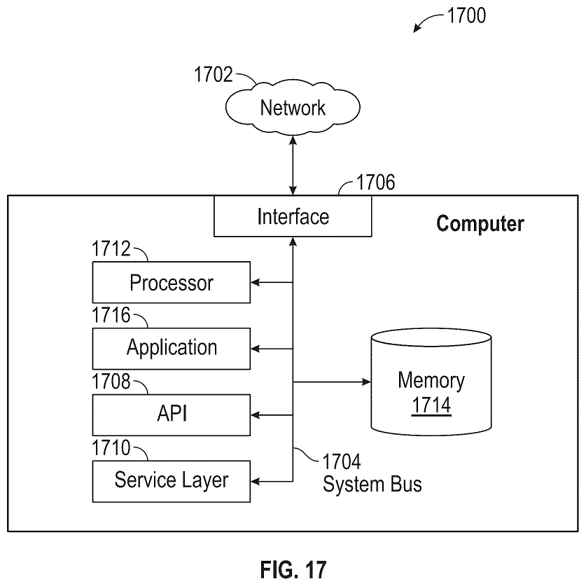

The well planning system may include dedicated software stored on a memory of a computer system, such as the computer system shown in FIG. 17. The wellbore plan may be informed by the best available information at the time of planning. This may include models encapsulating subterranean stress conditions, the trajectory of any existing wellbores (which may be desirable to avoid), and the existence of other drilling hazards, such as shallow gas pockets, over-pressure zones, and active fault planes.

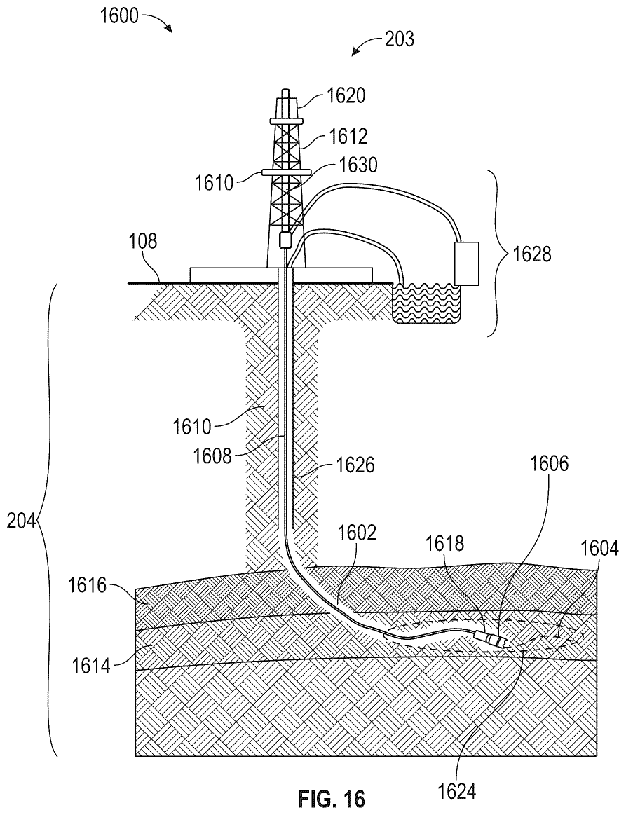

The wellbore path (1604, FIG. 16) may include a starting surface location of the wellbore, or a subsurface location within an existing wellbore, from which the wellbore (1602, FIG. 16) may be drilled. The wellbore path may further include a terminal location that may intersect with the previously located hydrocarbon reservoir (e.g., reservoir 114). The wellbore path may further still include wellbore geometry information such as wellbore diameter and inclination angle and when each of these change along the depth of the wellbore. If casing is used, the wellbore plan 316 may include casing type or casing depths. Furthermore, the wellbore plan may consider other engineering constraints such as the maximum wellbore curvature (dog-log) that a drillstring of a drilling system may tolerate and the maximum torque and drag values that the drilling system may tolerate. The wellbore plan may further define associated drilling parameters, such as the planned depths at which casing will be inserted to support the wellbore to prevent formation fluids entering the wellbore and the drilling mud weights (densities) and types that may be used during drilling of the wellbore.

In other embodiments, the calibrated geological model 314 may be input to a reservoir simulator 310. A reservoir simulator 310 comprises functionality for simulating the flow of fluids, including hydrocarbon fluids such as oil and gas, through a hydrocarbon reservoir composed of porous, permeable reservoir rocks in response to natural and anthropogenic pressure gradients. The reservoir simulator 310 may be used to predict changes in fluid flow, including fluid flow into well penetrating the reservoir as a result of planned well drilling, and fluid injection and extraction. For example, the reservoir simulator may be used to predict fluid-flow and production scenarios (e.g., production scenarios 320) including changes in hydrocarbon production rate that would result from the injection of water into the reservoir from wells around the reservoirs periphery.

The reservoir simulator 310 may use a geological model or reservoir model (e.g., calibrated geological model 314) that contains a digital description of the physical properties of the rocks as a function of position within the reservoir and the fluids within the pores of the porous, permeable reservoir rocks at a given time. In some embodiments, the digital description may be in the form of a dense 3D grid with the physical properties of the rocks and fluids defined at each node. In some embodiments, the 3D grid may be a cartesian grid, while in other embodiments the grid may be an irregular grid.

The physical properties of the rocks and fluids within the reservoir may be obtained from a variety of geological and geophysical sources. For example, remote sensing geophysical surveys, such as seismic surveys, gravity surveys, and active and passive source resistivity surveys, may be employed. In addition, data collected from well logs acquired in well penetrating the reservoir may be used to determine physical and petrophysical properties along the segment of the well trajectory traversing the reservoir. For example, porosity, permeability, density, seismic velocity, and resistivity may be measured along these segments of wellbore. In accordance with some embodiments, remote sensing geophysical surveys and physical and petrophysical properties determined from well logs may be combined to estimate physical and petrophysical properties for the entire reservoir simulation model grid.

Reservoir simulators solve a set of mathematical governing equations that represent the physical laws that govern fluid flow in porous, permeable media. For example, the flow of a single-phase slightly compressible oil with a constant viscosity and compressibility the equations capture Darcy's law, the continuity condition, and the equation of state.

Additional, and more complicated equations are required when more than one fluid, or more than one phase, e.g., liquid and gas, are present in the reservoir. Further, when the physical and petrophysical properties of the rocks and fluids vary as a function of position the governing equations may not be solved analytically and must instead be discretized into a grid of cells or blocks. The governing equations must then be solved by one of a variety of numerical methods, such as, without limitation, explicit or implicit finite-difference methods, explicit or implicit finite element methods, or discrete Galerkin methods.

The fluid flow and production scenarios (e.g., production scenarios 320) produced by the reservoir simulator 310 may then be used by the well planning system 300 to determine the wellbore plan 316. Using the wellbore plan the wellbore may be drilled (e.g., a drill wellbore 318 step in the well planning system 300.)

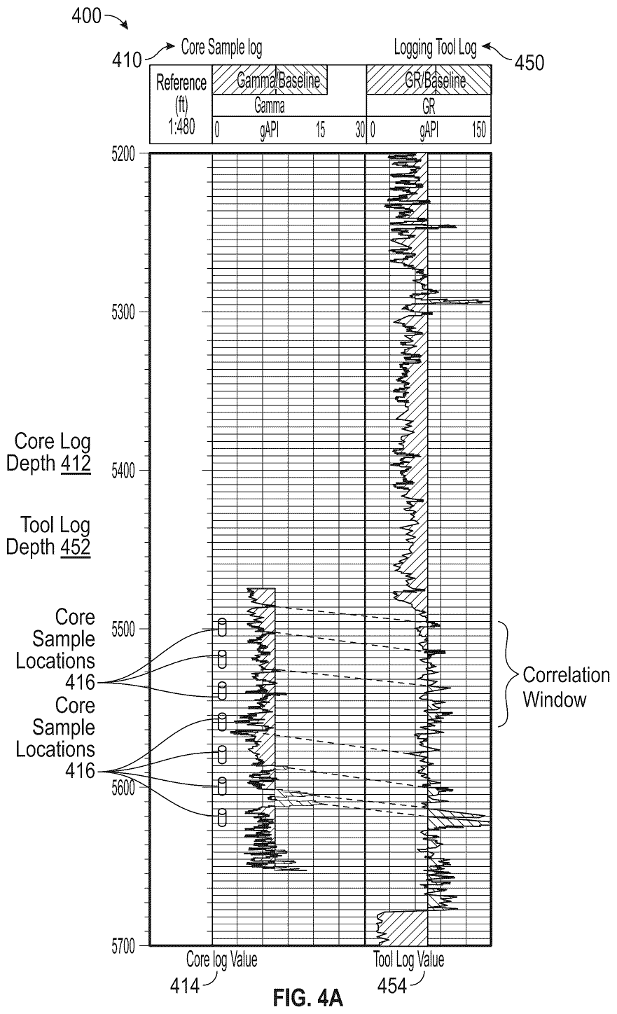

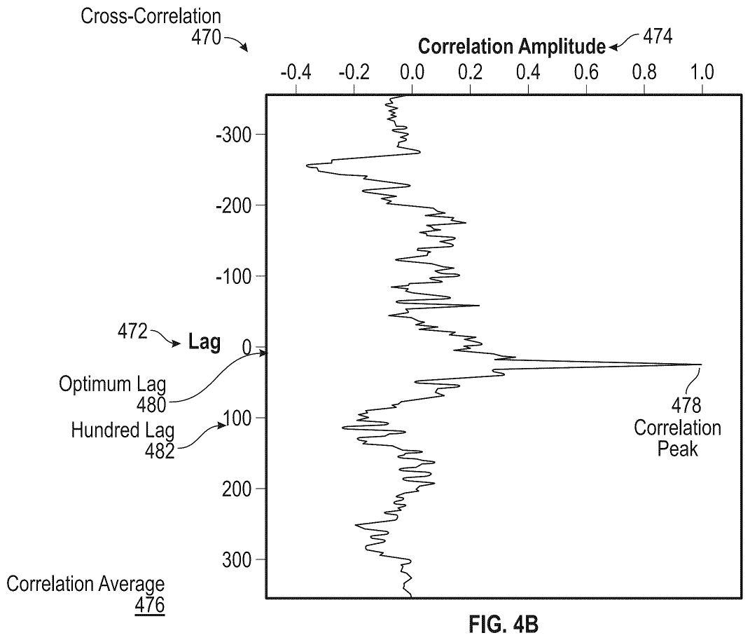

FIGS. 4A and 4B depict a comparison of a log from a core sample and a log from a logging tool. FIG. 4A shows a log comparison 400 of a core sample log 410 (Core GR) and a logging tool log 450 (Log GR). Core sample log 410 shows a core log depth 412 plotted on a vertical axis and a core log value 414 plotted on a horizontal axis. The core sample log may be acquired by a gamma ray (core sample bench test machine). In this case the core sample log may be referred to as a core GR (core gamma ray.) Core sample locations (e.g., a core sample location 416) are plotted on the core sample log. A correlation window 418 represents a depth-window surrounding a log event pair.

Logging tool log 450 shows a tool log depth 452 on a vertical axis and a tool log value 454 plotted on a horizontal axis. The logging tool log may be acquired by a gamma ray logging tool. In this case the logging tool log may be referred to as a log GR (log gamma ray). The gamma ray logging tool may be configured to acquire the gamma ray log upon receipt of a signal, such as a command signal, to begin logging. Furthermore, acquisition of the gamma ray log may commence after receipt of the signal to begin logging. The control system 122 or the control system 216 may send the command to begin logging. The core log depth 412 and the tool log depth 452 may correspond with a logging depth from one or more additional logging tools.

FIG. 4B shows a cross-correlation 470 between the core log value (the Core GR) and the tool log value (the Log GR) for a given offset/shift labeled as a lag 472. Lag 472 is the physical shift up or down relative to each other. The amplitude of the correlation (e.g., a correlation amplitude 474) shows a normalized dot product of the log values for each lag value. The correlation amplitude 474 sets the average of the correlations to zero (e.g., a correlation average 476) and the maximum of the correlations to one (e.g., a correlation peak 478). The dot product is the sum of the products of the corresponding entries of the two sequences of numbers. The amplitude shows how closely correlated are the core log value with the tool log value.

The peak (e.g., the correlation peak 478) shows the best alignment. The correlation peak 478 occurs with an optimum lag 480 that is shown between the zero lag (e.g., the correlation average 476) and a hundred lag 482. For each shift calculate the dot product. The graph shown in FIG. 4B may be generated by moving the target log (log to be shifted) with an incremental step (such as every 0.5 ft) and then calculating the correlation coefficient between the shifted log vs. the reference log. A peak correlation may be observed when the optimal bulk shift is used. The graph shown in FIG. 4B may be calculated as follows:

Step 1) lag (shift or offset) the core image up/down a certain amount (e.g., an offset of 1/64″) vs. the log plot.

Step 2) multiply the amplitudes (the core log value× the tool log value) for a set of depths. The quantity of depths (granularity) is directly proportional to the resolution of the resulting dot product.

Step 3) Add the products (the results of the multiplications) to get a correlation.

Step 4) Repeat the process beginning at step 1 for a different lag, e.g., 1/32″. The highest value sum indicates the best fit for the lag you selected in step 1. Note that values such as 1/64″ or 1/32″ are not to scale. 1/64″ may scale to, for example, two feet or ten feet in the actual well and/or core.

As shown in FIG. 4A, the peaks of the wireline logs appear to be shifted to slightly deeper depths than the core tool log. The shift can be quantitatively estimated using a cross-correlation function that quantifies the match between the two logs as a function of an applied depth shift between the two logs. The peak of the cross-correlation function, where the logs match one another to the greatest degree, may indicate an estimate of the actual difference between the depth scales of the two logs. The shift can be quantified by the correlation and the shift with the greatest degree thereby determines a bulk shift.

The bulk shift is not always sufficient to achieve the best match. The bulk shift is calculated using the entire available log, and the bulk shift is a single scalar shift of the entire log depth scale, vs. a variable shift calculated using depth windows spanning events in the log. The bulk shift is a non-variable displacement, whereas several variable shifts or variable stretching factors may affect the shift, for example the Yo-Yo effect, vibration, unloading, and damage discussed earlier. Detailed depth matching to account for the variable shift is done by manually picking the peaks and/or troughs on the two different logs and then matching peak to peak or trough to trough to form a set of matched depths. A depth shift table can be created using the set of depths correlating the depth on target log to the depth of the reference log. Besides the labor-intensive manual aspect of the procedure, the thick and homogeneous or extremely heterogeneous formations may not show peaks or troughs on logs. Furthermore, the depth shift table may not be easy to apply on core photos.

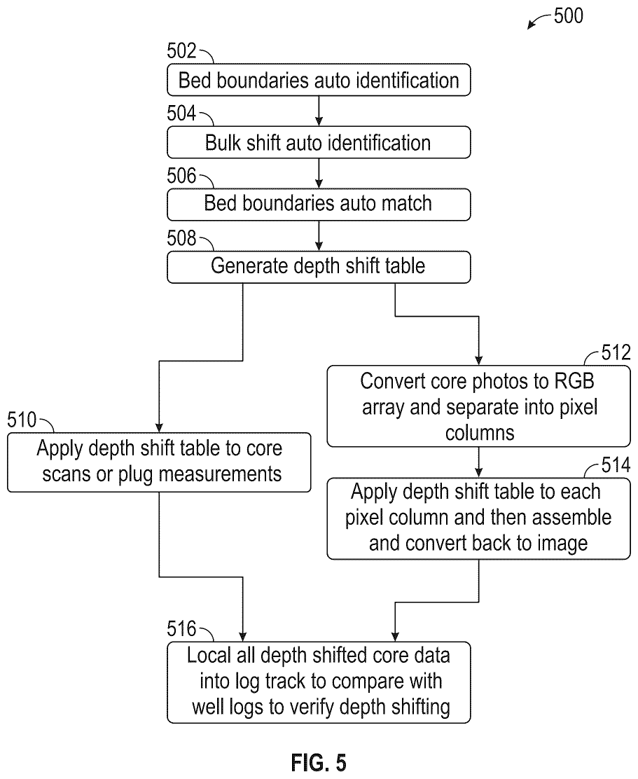

FIG. 5 shows a systematic workflow to automatically match the depth of core and well log depth to a best condition (i.e., a core log matching method 500). The workflow is generally applicable to any set of logs, core log to well log, different log runs, or different logs in the same run. The workflow involves automatically identifying a series of bed boundaries on both core gamma ray log and well gamma ray log through maximum variance method (an identify bed step 502).

Automatically may refer to the use of intelligent algorithms such as an algorithm that uses, for example, a Core GR log, a well log GR, and computing facilities that run a software written using a programming language such as Python®, a registered trademark of the Python Software Foundation. The algorithm identifies a series of events on the log curve and then matches them using a local maximum correlation standard. Commercially available software, programs, and/or programming languages may be used to perform the method. The citation of Python is not intended to be limiting, nor are the determinations intended to be limited to the commercially available software, program, and/or programming language. Any suitable software (e.g., custom-coded applications) providing similar functionality to that described may also be implemented without departing from the scope of the present disclosure.

Next, an optimal bulk shift between core gamma ray log and well gamma ray log is automatically identified to achieve the best correlation (an identify bulk step 504). Step 504 may determine a bulk-shift depth correction based on a first cost function such as a cross correlation or sum of absolute difference formed from the reference log, such as a core log (core GR), and the target log, such as a wireline log (log GR). The bulk offset may be a bulk shift depth correction. Applying the bulk-shift depth correction to the uncalibrated log (e.g., the target log) may form a bulk-shifted log. Applying the bulk-shift depth correction to the uncalibrated log (e.g., the target log) to form a bulk-shifted log may be performed automatically.

Continuing with FIG. 5, in a match bed step 506, the bed boundaries between core gamma ray log and well gamma ray log are automatically matched using an optimization algorithm that minimizes the bed layer boundary shift variance and mapping stretch and maximizes the curves correlation. Step 506 may identify log event pairs, such as bed layer pairs, that may be identified on both the reference log, such as the core GR, and the target log, such as the log GR. For example, a bed layer boundary identified on the core GR may be paired with a bed layer boundary on the log GR.

Once the best depth match is achieved, a depth shift table is generated (step 508), and applied to all core scans or core plug measurements (step 510). The log event pairs identified on the reference log (e.g., core GR) and on the target log (e.g., log GR) may be offset or shifted in depth from one another. The log event on the reference log may be shifted from the corresponding event on the target log and/or the log event on the target log may be shifted from the corresponding log event on the reference log. Determining a shift (e.g., a local-shift depth) for each of a set of log event pairs may be formed into a depth shift table. A local-shift depth correction may be determined from the depth shift table. The local offset may be a local-shift depth correction. A local-shift depth correction table may be formed from the local-shift depth correction for the log event pairs. In this manner, the local-shift depth correction may be determined for each of the log event pairs using a second cost function formed from the reference log and the bulk-shifted log over a depth-window surrounding the log event pair. The local-shift depth correction for each log event pair may be compiled to form a local-shift depth correction table. Applying the local-shift depth correction to the uncalibrated geological model may form a calibrated geological model.

For core photo data type, it is first converted to an RGB (red-green-blue) array and then each vertical pixel column is separated (a convert photo step 512). The depth shift table is applied to each column. An RGB array is a data array that defines red, green, and blue color components of each pixel, e.g., an RGB pixel array has at least one of each of a red value, a green value, and a blue value. The pixels may be separated in their colors to form columns, e.g., a red value column, a green value column, and a blue value column. The local-shift depth correction table may be applied to each of the red value column, the green value column, and the blue value column. After depth shifting each column, all columns are assembled together and converted back to an image (a convert image step 514).

At this stage, all core data including core plugs/scans/photos is loaded into well log tracks and compared with well logs to quality check and verify that all depths are matching to the best condition (a verify shift step 516).

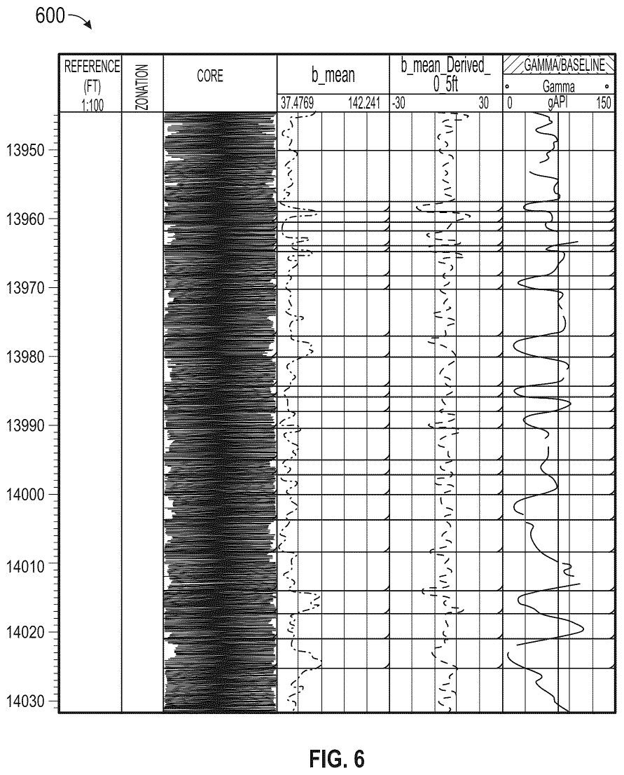

In some embodiments, a gamma-ray log is generated from gamma-ray logging data. A gamma-ray log may be a data record that is obtained using a gamma-ray logging tool. Likewise, the gamma-ray logging data bay also be monitored at a well continuously in real-time. An example of a gamma-ray log is illustrated in FIG. 6, which shows a gamma ray log 600 in accordance with one or more embodiments. Gamma ray log 600 may be used as a log for performing the identify bed step 502 of core log matching method 500 to automatically identify bed boundaries on logs or scans.

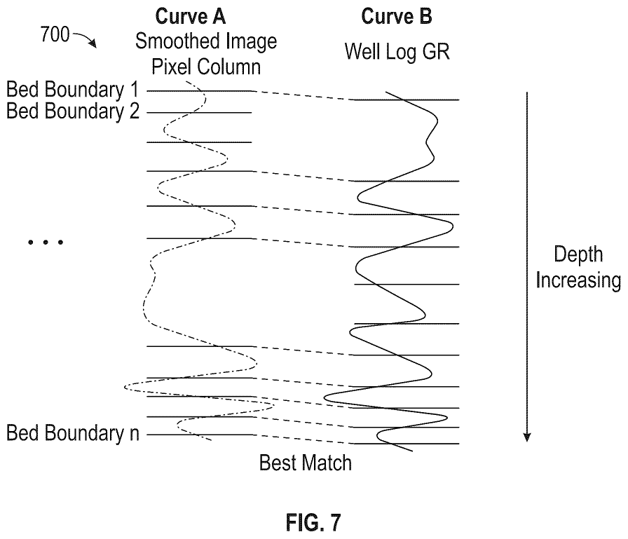

In some embodiments, a best match of two curves is generated from gamma-ray or other logging data. Curves of data identifying bed boundaries for various depths may be data records obtained using a gamma-ray logging tool or other logging tools. An example of a comparison between two curves is illustrated in FIG. 7, which shows a bed match 700 in accordance with one or more embodiments. Bed match 700 compares a first set of bed boundaries numbering 1 through n (curve A, such as a smoothed image pixel column) with a second set of bed boundaries numbering 1 through n (curve B, such as a well log GR). Bed match 700 may illustrate the match bed step 506 of core log matching method 500. Bed match 700 is an example illustration of automatically matching the bed boundaries between core gamma ray log and well gamma ray log by using an optimization algorithm that minimizes the bed boundary shift variance and maximizes the curves correlation.

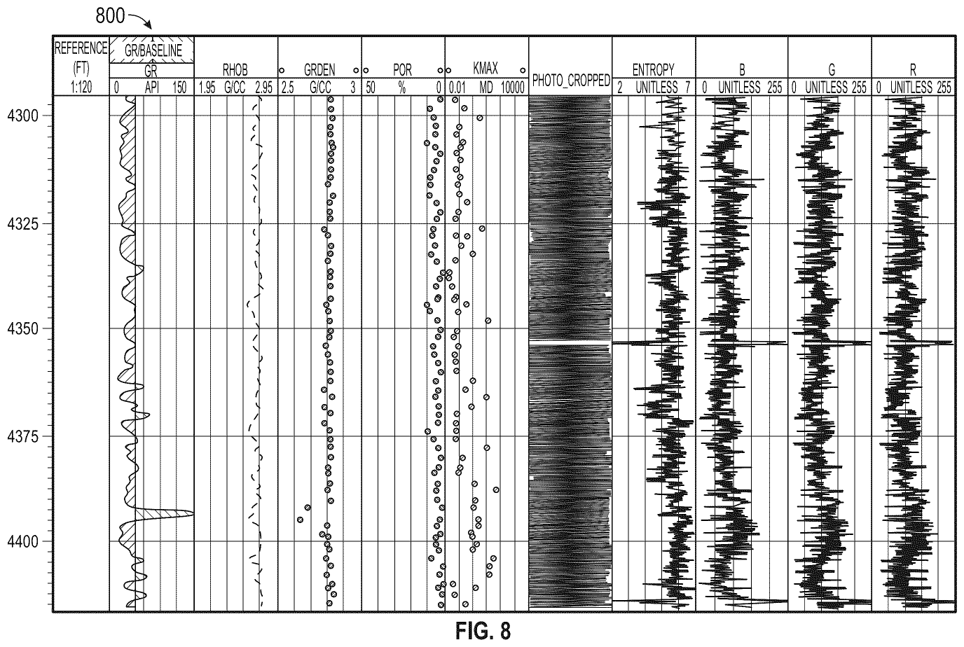

In some embodiments a depth shift table may be applied to core photo data. An example of an application of depth shifts to vertical pixel columns is illustrated in FIG. 8, which shows a color shift 800 in accordance with one or more embodiments. For example, core photo data may be converted to an RGB array. Each color of the array may be separated into separate columns such as vertical columns of R, G, and B. The depth shift table values may then be applied to each of the R, G, and B columns. Color shift 800 may illustrate the apply table step 510 of core log matching method 500. Color shift 800 is an example illustration of applying the depth shift table to core photo data, including converting core photo to an RGB array, separating each vertical pixel column, and then applying the depth shift table to each column.

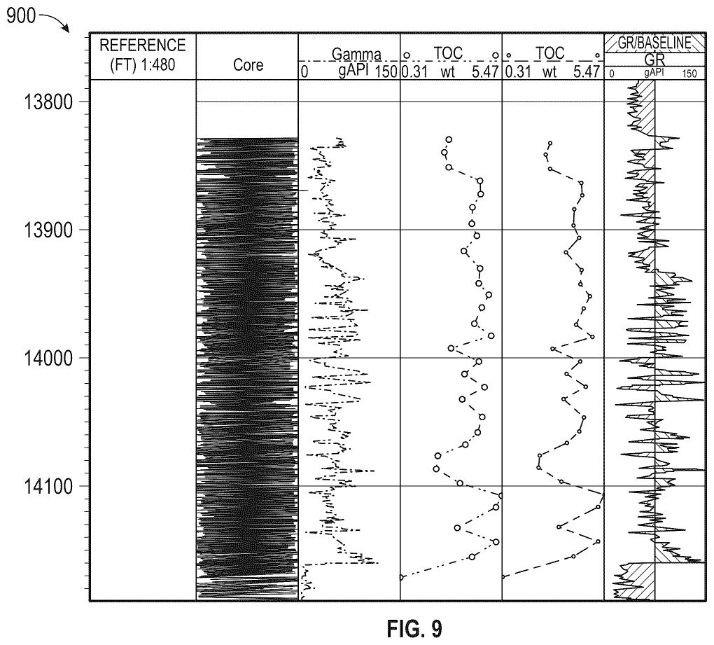

In some embodiments all core data such as core plugs data, core scans data, and core photos data may be loaded into well log tracks and compared with well logs. An example of a loading of core data into well log tracks is illustrated in FIG. 9, which shows well log tracks (e.g., a well log track 900) in accordance with one or more embodiments. For example, well log tracks are shown with core data, gamma data, total organic carbon (TOC) data, and gamma-ray baseline data. Well log track 900 may illustrate the verify shift step 516 of core log matching method 500. Well log track 900 is an example illustration of loading all core data including core plugs data, core scans data, and core photos data into well log tracks and comparing with well logs.

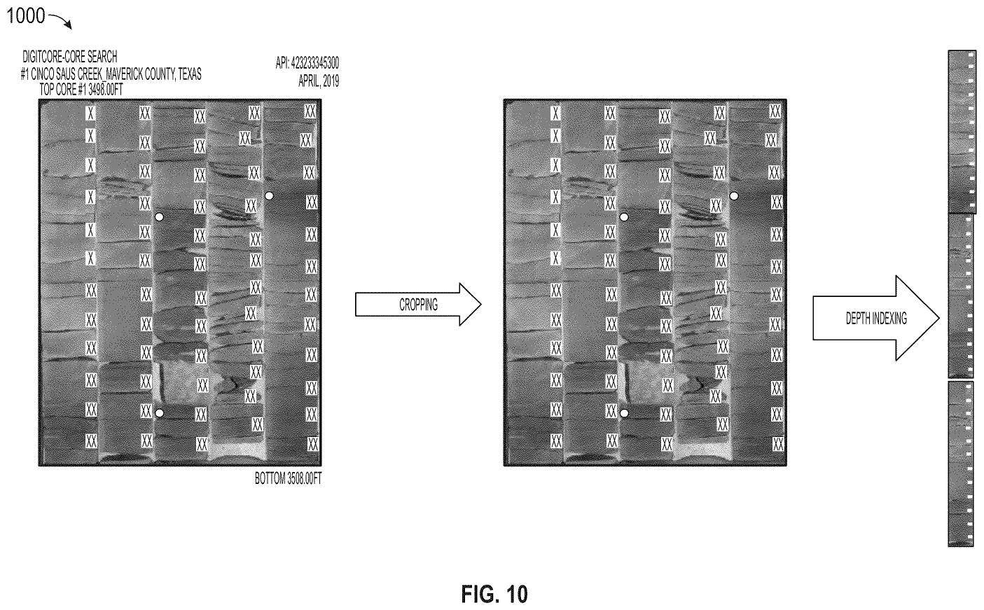

In some embodiments the core plugs photos may be used to determine subsurface measurements of rock properties from core logs and well logs indexing the photos by depth. Depth indexing may include cropping the core photos. An example of cropping the core photos and performing depth indexing of the core photos is illustrated in FIG. 10, which shows core photos (e.g., a core photo 1000) in accordance with one or more embodiments. For example, core photos are shown in a cropped condition and in a depth indexing condition. Core photo 1000 may illustrate the convert photo step 512 and the convert image step 514 of core log matching method 500.

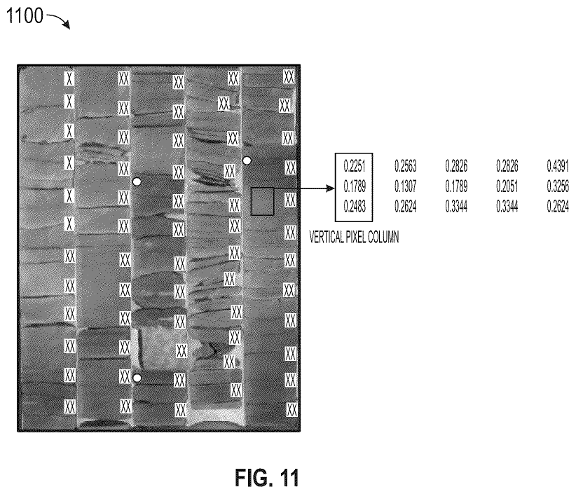

In some embodiments the core plug photos may be used to determine depths of rock properties by converting the core plug photos to an RGB array. Using core plug photos to determine depths of rock properties may include separating each vertical pixel column. An example of converting a core photo to an RGV array and then separating each vertical pixel column is illustrated in FIG. 11, which shows pixel array (e.g., a pixel array 1100) in accordance with one or more embodiments. For example, a pixel array is show for a region of a core photo and the pixels are arranged in a column. Pixel array 1100 may illustrate the convert photo step 512.

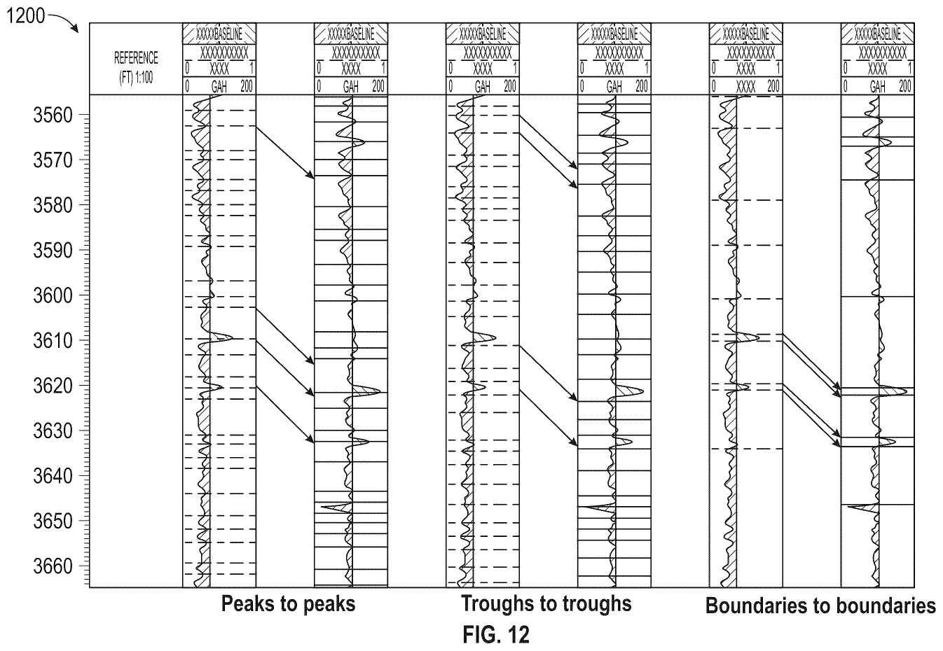

FIGS. 12-15 show examples in accordance with one or more embodiments. FIG. 12 shows matching events between log curves. For example, detecting all different types of events on both core gamma ray and well log gamma ray and matching those events to generate a depth shift table. Arrows in FIG. 12 illustrate how different types of events are matched (e.g., event match 1200) to generate anchoring points in a depth shift table. Event match 1200 may illustrate the match bed step 506 of core log matching method 500. Example criteria to match events between two logs may include:
1) Matching the same type of events such as peak to peak, trough to trough, and bed boundary to bed boundary;
2) Anchoring depth points within a given depth correspondence limit such as 2′ (feet) after bulk shift;
3) Meeting a predetermined correlation criteria. For example, points in a sliding window (e.g., a depth-window sized at 3′ or 5′) surrounding the log event pair (e.g., anchoring points) may meet a correlation criteria such as an R2>0.75; and
4) Conforming anchoring lines to corresponding anchoring lines and eliminating outliers.

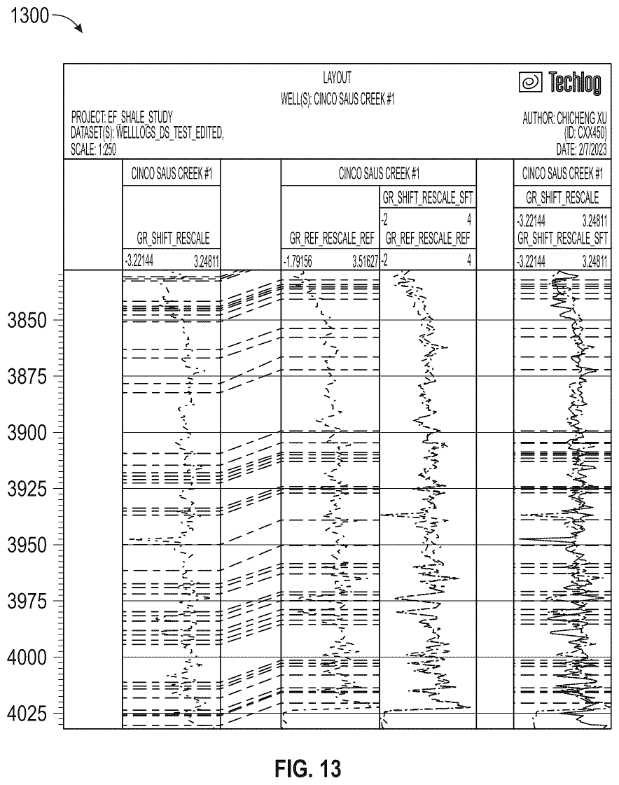

FIG. 13 shows an example of depth shift table validation from a field location. FIG. 13 shows a field example depth shift table 1300 showing the depth shift table in a depth shift window. The automatically-generated depth shift table may be loaded into the depth shift window and checked for how well the shift logs aligned with the reference log. This step allows users to quality control the automatic depth shifting results and affords users the opportunity to modify the results if the user wants to add or delete some of the anchoring point pairs. This check of quality control may be a form of validation and/or verification of the results of the core log matching method 500. Field example depth shift table 1300 may illustrate the verify shift step 516 of core log matching method 500.

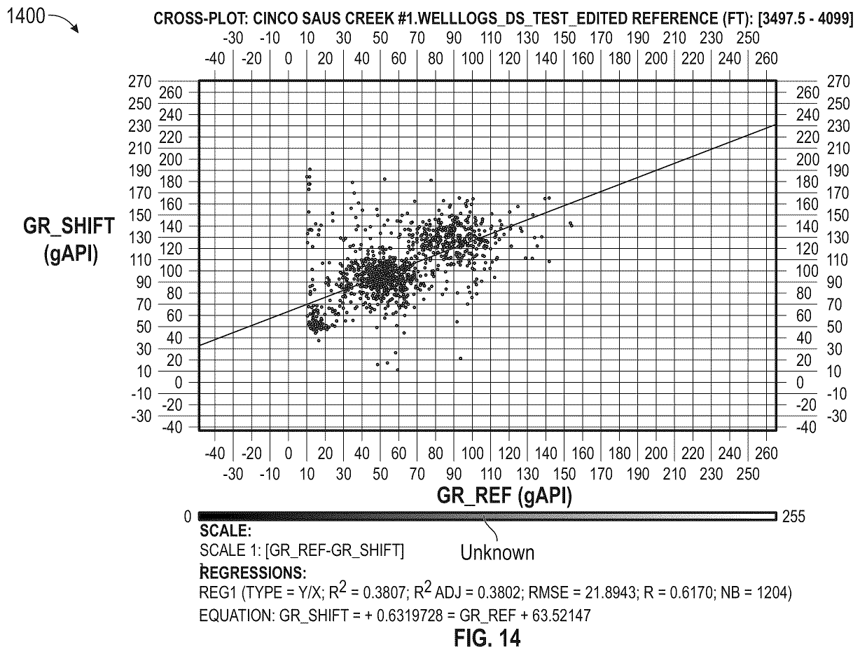

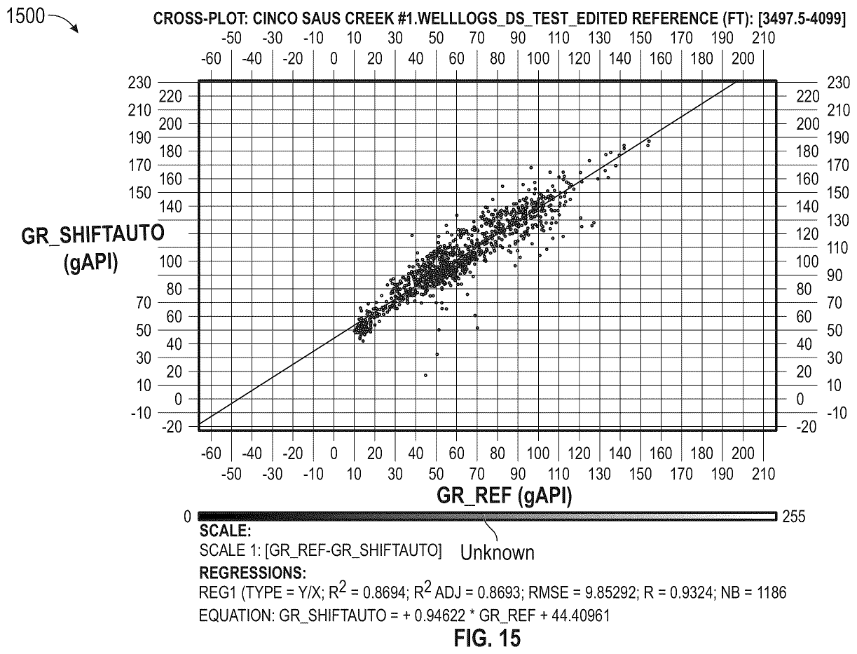

A second validation would be checking the correlation enhancement before and after depth shifting. FIG. 14 shows the correlation between core gamma ray and well log gamma ray before automatic depth shifting (e.g., a pre-shift correlation 1400). In FIG. 14 a correlation value example may be R²=0.38. FIG. 15 shows the correlation between core gamma ray and well log gamma ray after automatic depth shifting (e.g., a post-shift correlation 1500). In FIG. 15 a correlation value example may be R²=0.87.

FIG. 16 depicts a wellbore trajectory in accordance with one or more embodiments. The planned wellbore trajectory (e.g., a wellbore trajectory 1600) contained in the well drilling plan (e.g., wellbore plan 316) may then be transferred to a drilling system 203 such that the wellbore path 1604, guided by the planned wellbore trajectory, may be drilled as illustrated in FIG. 16 in accordance with one or more embodiments. Although the drilling system 203 shown in FIG. 16 is used to drill the wellbore 1602 on land, the drilling system may also be a marine wellbore drilling system. The example of the drilling system shown in FIG. 16 is not meant to limit the present disclosure.

As shown in FIG. 16, the wellbore 1602 may be drilled using a drill rig that may be situated on a land drill site, an offshore platform, such as a jack-up rig, a semi-submersible rig, or a drill ship. The drill rig may be equipped with a hoisting system, such as a derrick 1612, which can raise or lower the drillstring 1608 and other tools required to drill the wellbore. The drillstring 1608 may include one or more drill pipes connected to form conduit and a bottom hole assembly (e.g., a BHA 1618) disposed at the distal end of the drillstring 1608. The BHA 1618 may include a drill bit 1606 to cut into rock 1610, including cap rock 1616. The BHA 1618 may further include measurement tools, such as a measurement-while-drilling (MWD) tool and logging-while-drilling (LWD) tool. MWD tools may include sensors and hardware to measure downhole drilling parameters, such as the azimuth and inclination of the drill bit 1606, the weight-on-bit, and the torque. The LWD measurements may include sensors, such as resistivity, gamma ray, and neutron density sensors, to characterize the rock 1610 surrounding the wellbore. Both MWD and LWD measurements may be transmitted to the surface using any suitable telemetry system known in the art, such as a mud-pulse or by wired-drill pipe. Note that the shift obtained from the gamma-ray comparison can also be applied to other wireline logs or logs from other logging tools, i.e., other eLogs.

To start drilling, or “spudding in,” the wellbore, the hoisting system lowers the drillstring 1608 suspended from the derrick 1612 towards the planned surface location of the wellbore. An engine, such as a diesel engine, may be used to supply power to the top drive 1620 to rotate the drillstring 1608 via the drive shaft 1630. The weight of the drillstring 1608 combined with the rotational motion enables the drill bit 1606 to bore the wellbore.

The near-surface of the subterranean region of interest is typically made up of loose or soft sediment or rock, therefore a relatively large diameter casing (e.g., casing 1626), also known as base pipe or conductor casing, is often put in place while drilling to stabilize and isolate the wellbore. At the top of the base pipe is the wellhead, which serves to provide pressure control through a series of spools, valves, or adapters. Once near-surface drilling has begun, water or drill fluid may be used to force the base pipe into place using a pumping system until the wellhead is situated just above the surface 108 of the earth.

Drilling may continue without any casing 1626 once deeper or more compact rock is reached. While drilling, a drilling mud system 1628 may pump drilling mud from a mud tank on the surface of the earth through the drill pipe. Drilling mud serves various purposes, including pressure equalization, removal of rock cuttings, and drill bit cooling and lubrication.

At planned depth intervals, drilling may be paused and the drillstring 1608 withdrawn from the wellbore 1602. Sections of casing 1626 may be connected and inserted and cemented into the wellbore. Casing string may be cemented in place by pumping cement and mud, separated by a cementing plug, from the surface 108 through the drill pipe. The cementing plug and drilling mud force the cement through the drill pipe and into the annular space between the casing 1626 and the wall of the wellbore. Once the cement cures, drilling may recommence. The drilling process is often performed in several stages. Therefore, the drilling and casing cycle may be repeated more than once, depending on the depth of the wellbore and the pressure on the walls of the wellbore from surrounding rock (e.g., rock 1610).

Due to the high pressures experienced by deep wellbores, a blowout preventer (BOP) may be installed at the wellhead to protect the rig and environment from unplanned oil or gas releases. As the wellbore becomes deeper, both successively smaller drill bits and smaller casing string may be used. Drilling deviated or horizontal wellbores may require specialized drill bits or BHA 1618.

The drilling system 203 may be disposed at and communicate with other systems in the well environment. The drilling system may control at least a portion of a drilling operation by providing controls to various components of the drilling operation. In one or more embodiments, the system may receive data from one or more sensors arranged to measure controllable parameters of the drilling operation. As a non-limiting example, sensors may be arranged to measure weight-on-bit, drill rotational speed (RPM), flow rate of the mud pumps (GPM), and rate of penetration of the drilling operation (ROP). Each sensor may be positioned or configured to measure a desired physical stimulus. Drilling may be considered complete when a drilling target 1624 within the hydrocarbon reservoir 1614 is reached or the presence of hydrocarbons is established.

In some embodiments the well planning system 300, the seismic processing system 218, and the seismic interpretation workstation 221 may each include a computer system such as the computer system shown in FIG. 17.

FIG. 17 is a block diagram of a computer system (e.g., a computer 1700) used to provide computational functionalities associated with described algorithms, methods, functions, processes, flows, and procedures as described in the instant disclosure, according to an implementation. The illustrated computer is intended to encompass any computing device such as a high performance computing (HPC) device, a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer may include a computer that includes an input device, such as a keypad, keyboard, touch screen, or other device that can accept user information, and an output device that conveys information associated with the operation of the computer, including digital data, visual, or audio information (or a combination of information), or a GUI.

The computer (1700) can serve in a role as a client, network component, a server, a database or other persistency, or any other component (or a combination of roles) of a computer system for performing the subject matter described in the instant disclosure. The illustrated computer is communicably coupled with a network (1702). In some implementations, one or more components of the computer may be configured to operate within environments, including cloud-computing-based, local, global, or other environment (or a combination of environments).

At a high level, the computer (1700) is an electronic computing device operable to receive, transmit, process, store, or manage data and information associated with the described subject matter. According to some implementations, the computer (1700) may also include or be communicably coupled with an application server, e-mail server, web server, caching server, streaming data server, business intelligence (BI) server, or other server (or a combination of servers).

The computer (1700) can receive requests over the network (1702) from a client application (for example, executing on another computer) and responding to the received requests by processing the said requests in an appropriate software application. In addition, requests may also be sent to the computer (1700) from internal users (for example, from a command console or by other appropriate access method), external or third-parties, other automated applications, as well as any other appropriate entities, individuals, systems, or computers.

Each of the components of the computer (1700) can communicate using a system bus (1704). In some implementations, any or all of the components of the computer (1700), both hardware or software (or a combination of hardware and software), may interface with each other or with an interface (1706) (or a combination of both) over the system bus using an application programming interface (an API 1708) or a service layer (1710) (or a combination of the API and the service layer. The API may include specifications for routines, data structures, and object classes. The API may be either computer-language independent or dependent and refer to a complete interface, a single function, or even a set of APIs.

The service layer (1710) provides software services to the computer (1700) or other components (whether or not illustrated) that are communicably coupled to the computer. The functionality of the computer may be accessible for all service consumers using this service layer. Software services, such as those provided by the service layer, provide reusable, defined business functionalities through a defined interface. For example, the interface may be software written in JAVA, C++, or other suitable language providing data in extensible markup language (XML) format or other suitable format. While illustrated as an integrated component of the computer (1700), alternative implementations may illustrate the API or the service layer as stand-alone components in relation to other components of the computer (1700) or other components (whether or not illustrated) that are communicably coupled to the computer. Moreover, any or all parts of the API or the service layer may be implemented as child or sub-modules of another software module, enterprise application, or hardware module without departing from the scope of this disclosure.

The computer (1700) includes the interface (1706). Although illustrated as a single interface in FIG. 17, two or more of interface 1706 may be used according to particular needs, desires, or particular implementations of the computer. The interface is used by the computer (1700) for communicating with other systems in a distributed environment. The other systems are connected to the network (1702). Generally, the interface includes logic encoded in software or hardware (or a combination of software and hardware) and operable to communicate with the network. More specifically, the interface may include software supporting one or more communication protocols associated with communications such that the network or the hardware of the interface is operable to communicate physical signals within and outside of the computer.

The computer (1700) includes at least one of a computer processor (1712). Although illustrated as a single computer processor in FIG. 17, two or more processors may be used according to particular needs, desires, or particular implementations of the computer. Generally, the computer processor executes instructions and manipulates data to perform the operations of the computer (1700) and any algorithms, methods, functions, processes, flows, and procedures as described in the instant disclosure.

The computer (1700) also includes a memory (1714) that holds data for the computer (1700) or other components (or a combination of both) that may be connected to the network (1702). For example, memory may be a database storing data consistent with this disclosure. Although illustrated as a single memory in FIG. 17, two or more memories may be used according to particular needs, desires, or particular implementations of the computer and the described functionality. While the memory is illustrated as an integral component of the computer, in alternative implementations, the memory may be external to the computer.

An application (1716) is an algorithmic software engine providing functionality according to particular needs, desires, or particular implementations of the computer (1700), particularly with respect to functionality described in this disclosure. For example, the application can serve as one or more components, modules, applications, etc. Further, although illustrated as a single application, the application may be implemented as multiple applications on the computer. In addition, although illustrated as integral to the computer, in alternative implementations, the application may be external to the computer.

There may be any number of the computer (1700) associated with, or external to, a computer system containing computer, each computer communicating over network (1702). Further, the term “client,” “user,” and other appropriate terminology may be used interchangeably as appropriate without departing from the scope of this disclosure. Moreover, this disclosure contemplates that many users may use one of computer (1700), or that one user may use multiple computers.

In some embodiments, the computer (1700) is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, cloud computing system may operate according to one or more service models, such as infrastructure as a service (IaaS), platform as a service (PaaS), software as a service (SaaS), mobile “backend” as a service (MBaaS), serverless computing, artificial intelligence (AI) as a service (AIaaS), and/or function as a service (FaaS).

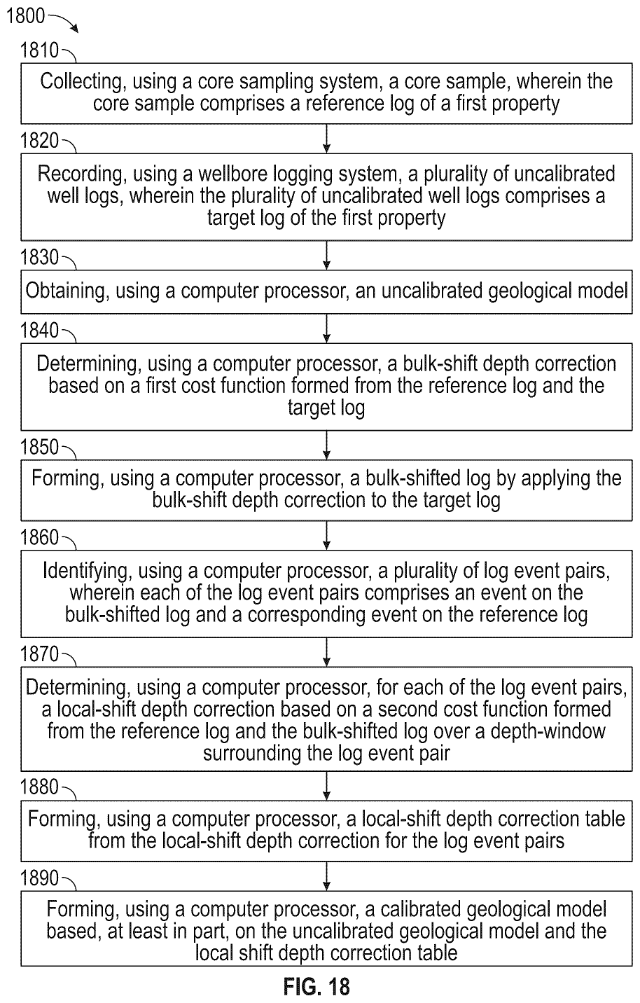

FIG. 18 shows a flowchart in accordance with one or more embodiments. Specifically, FIG. 18 describes a general method using a core sampling system to collect a core sample, record uncalibrated well logs, and form calibrated well logs of a wellbore (e.g., a calibration method 1800). One or more blocks in FIG. 18 may be performed by one or more components (e.g., control system 216, seismic processing system 218, and/or seismic interpretation workstation 221) as described in FIGS. 1-17. While the various blocks in FIG. 18 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

At step 1810, one or more commands are transmitted to one or more well logging tools in a wellbore and/or to a core sampling system in accordance with one or more embodiments. For example, an optical fiber cable may be coupled to a control system, well logging tools, and/or core sampling tools. Any number of commands may be sent to any number of well logging tools and/or core sampling tools. The core sampling system, upon receipt of the command, may then collect a core sample. The core sample may have a reference log of a first property. The core sample may have a first property of, for example, a gamma-ray emission.

At step 1820, well log data is obtained from one or more logging tools in response to one or more commands in accordance with one or more embodiments. The wellbore logging system records the well log data. In particular, method step may include recording, using a wellbore logging system, a plurality of uncalibrated well logs. The uncalibrated well logs may have a target log of the first property. The uncalibrated well logs may have the first property of, for example, the bed layer boundary. The well log data may be correlated with a time and data stamp to create a history. The well log data may further include a depth at which the data was recorded. The well log data may be obtained from the logging tool(s) in response to receipt of a transmitted command(s). The control system 216, seismic processing system 218, and/or seismic interpretation workstation 221 may transmit the command. The well log data are generated using the logging tool(s).

At step 1830, in accordance with one or more embodiments, the method may include using a computer processor to obtain an uncalibrated geological model. At step 1840 the computer processor may determine a bulk-shift depth correction based on a first cost function formed from the reference log and the target log. For example, referring back to FIG. 4A, FIG. 4A shows a typical dataset with gamma ray (GR) from both core and log. Given a relatively large depth offset between the target log and the reference log, the computer processor may first perform an optimal bulk shift between these two logs. The target log may be shifted within a large window (e.g., ±50 ft) every sampling rate (e.g., 0.5 ft) to identify maximum overall correlation between these two logs. FIG. 4B shows the peak correlation was found to be achieved at +10 ft bulk shift. At step 1850 the computer processor may be used to form a bulk-shifted log by applying the bulk-shift depth correction to the target log. This bulk depth shift determined in step 1840 may be applied to the target log before further fine-tuning depth matching.

At step 1860, the computer processor may be used to identify a log event pair and/or more than one log event pair. For example, each of the log event pairs may have an event on the bulk-shifted log and a corresponding event on the reference log. Various types of events on well logs are defined as follows: Peaks: local maximum of a curve; Troughs: local minimum of a curve; Bed boundaries: max variance along a sliding window that may be on a ramping up trend (gradient>0) or a ramping down trend (gradient<0). FIG. 12 shows a field example of identifying the four different events (peak, trough, bed ramping up boundary, bed ramping down boundary) on both core gamma ray and well log gamma ray. For example, the event on the bulk-shifted log may be an extremum of a depth derivative of the first property. The extremum of the depth derivative may be determined using a maximum variance method.

At step 1870, the method may include using the computer processor to determine, for each of the log event pairs, a local-shift depth correction based on a second cost function. The second cost function may be formed from the reference log and the bulk-shifted log over a depth-window surrounding the log event pair. After detecting all different types of events on both core gamma ray and well log gamma ray, the computer processor may match those events to generate a depth shift table. The criteria to match events between two logs may include the example criteria described in FIG. 12 and the associated description. The second cost function may be, for example, a cross-correlation.

At step 1880, the method may include using the computer processor to form a local-shift depth correction table from the local-shift depth correction for the log event pairs. The computer processor may use the events as matched in step 1870 according to all the criteria, the paired depth of each event may be output as a depth shift table dataset.

At step 1890, the method may include using the computer processor to form a calibrated geological model based, at least in part, on the uncalibrated geological model and the local-shift depth correction table. As shown in FIG. 13 and described in the associated description, the computer processor may load the automatically-generated depth shift table into the depth shift window and check how well the shift logs aligned with the reference log. The second validation may be performed as shown in FIG. 14 and FIG. 15 and described in the associated descriptions.

Forming the calibrated geological model may include, for example, taking a photographic image of each core sample, each image having multiple pixels, and then depth shifting each pixel. Forming the calibrated geological model may include validating the local-shift depth correction table. Forming the calibrated geological model may include converting a core photo into an RGB pixel array comprising a red value, a green value, and a blue value, then separating each vertical pixel column into a red value column, a green value column, and a blue value column, and then applying the local-shift depth correction table to each of the red value column, the green value column, and the blue value column.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims. In the claims, means-plus-function clauses are intended to cover the structures described herein as performing the recited function and not only structural equivalents, but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures. It is the express intention of the applicant not to invoke 35 U.S.C. § 112 (f) for any limitations of any of the claims herein, except for those in which the claim expressly uses the words ‘means for’ together with an associated function.

What is claimed:

1. A method, comprising:
collecting, using a core sampling system, a core sample, wherein the core sample comprises a reference log of a first property;
recording, using a wellbore logging system, a plurality of uncalibrated well logs, wherein the plurality of uncalibrated well logs comprises a target log of the first property; and
using a computer processor:
obtaining an uncalibrated geological model,
determining a bulk-shift depth correction based on a first cost function formed from the reference log and the target log,
forming a bulk-shifted log by applying the bulk-shift depth correction to the target log,
identifying a plurality of log event pairs, wherein each of the log event pairs comprises an event on the bulk-shifted log and a corresponding event on the reference log,
determining, for each of the log event pairs, a local-shift depth correction based on a second cost function formed from the reference log and the bulk-shifted log over a depth-window surrounding the log event pair,
forming a local-shift depth correction table from the local-shift depth correction for the log event pairs, and
forming a calibrated geological model based, at least in part, on the uncalibrated geological model and the local-shift depth correction table.

2. The method of claim 1, further comprising:
identifying, using an interpretation workstation, a drilling target based, at least in part, on the calibrated geological model; and
planning, using a well planning system, a planned wellbore trajectory to intersect the drilling target.

3. The method of claim 2, further comprising drilling, using a drilling system, a wellbore guided by the planned wellbore trajectory.

4. The method of claim 1, wherein the first property is gamma-ray emission.

5. The method of claim 1, wherein the second cost function comprises a cross-correlation.

6. The method of claim 1, wherein the event comprises an extremum of a depth derivative of the first property.

7. The method of claim 6, wherein the extremum of the depth derivative is determined using a maximum variance method.

8. The method of claim 1, wherein forming the calibrated geological model comprises:
taking a photographic image of each core sample, wherein each image comprises a plurality of pixels; and
depth shifting each pixel.

9. The method of claim 1, wherein forming the calibrated geological model further comprises validating the local-shift depth correction table.

10. The method of claim 8, wherein forming the calibrated geological model comprises:
converting a core photo into an RGB pixel array comprising a red value, a green value, and a blue value;
separating each vertical pixel column into a red value column, a green value column, and a blue value column; and
applying the local-shift depth correction table to each of the red value column, the green value column, and the blue value column.

11. The method of claim 10, further comprising:
assembling the red value column, the green value column, and the blue value column, and
converting the red value column, the green value column, and the blue value column back into a depth-shifted core image.

12. A system, comprising:
a core sampling system configured to collect a core sample, wherein the core sample comprises a reference log of a first property;
a wellbore logging system configured to record a plurality of uncalibrated well logs, wherein the plurality of uncalibrated well logs comprises a target log of the first property; and
a computer processor, configured to:
obtain an uncalibrated geological model,
determine a bulk-shift depth correction based on a first cost function formed from the reference log and the target log,
form a bulk-shifted log by applying the bulk-shift depth correction to the target log,
identify a plurality of log event pairs, wherein each of the log event pairs comprises an event on the bulk-shifted log and a corresponding event on the reference log,
determine, for each of the log event pairs, a local-shift depth correction based on a second cost function formed from the reference log and the bulk-shifted log over a depth-window surrounding the log event pair,
form a local-shift depth correction table from the local-shift depth correction for the log event pairs, and
form a calibrated geological model based, at least in part, on the uncalibrated geological model and the local-shift depth correction table.

13. The system of claim 12, further comprising:
an interpretation workstation, configured to identify a drilling target in the calibrated geological model; and
a well planning system, configured to plan a planned wellbore trajectory to intersect the drilling target.

14. The system of claim 13, further comprising a drilling system configured to drill a wellbore guided by the planned wellbore trajectory.

15. The system of claim 12, wherein the first property is gamma-ray emission.

16. The system of claim 12, wherein the second cost function comprises a cross-correlation.

17. The system of claim 12, wherein forming the calibrated geological model comprises:
taking a photographic image of each core sample, wherein each image comprises a plurality of pixels; and
depth shifting each pixel.

18. The system of claim 12, wherein forming the calibrated geological model further comprises validating the local-shift depth correction table.

19. The system of claim 12, wherein forming the calibrated geological model comprises:
converting a core photo into an RGB pixel array comprising a red value, a green value, and a blue value;
separating each vertical pixel column into a red value column, a green value column, and a blue value column; and
applying the local-shift depth correction table to each of the red value column, the green value column, and the blue value column.

20. The system of claim 19, further comprising:
assembling the red value column, the green value column, and the blue value column, and
converting the red value column, the green value column, and the blue value column back into a depth-shifted core image.
